(19) United States
(12) Patent Application Publication
(10) Pub. No.: US 2022/0088234 A1
(43) Pub. Date: Mar. 24, 2022

(54) AIR-EXCHANGING SANITATION APPARATUS AND METHOD

(71) Applicant: Nuytkens Industries LLC, Franklin, MA (US)

(72) Inventors: Peter R. Nuytkens, Franklin, MA (US); Mark H. Nuytkens, Mystic, CT (US); Amy R. Nuytkens, Newport, RI (US); Margaret H. Nuytkens, Franklin, MA (US)

(21) Appl. No.: 17/482,274

(22) Filed: Sep. 22, 2021

Related U.S. Application Data
(60) Provisional application No. 63/081,499, filed on Sep. 22, 2020.

(51) Int. Cl. A61L 2/00; A61L 2/10; A61L 9/20

(52) U.S. Cl. CPC A61L 2/0047; A61L 2/10; A61L 9/20; A61L 2/0052; A61L 2209/212; A61L 2202/14; A61L 2202/16; A61L 2209/111; A61L 2202/11

(57) ABSTRACT

Described herein is a low-form table-top air sanitizer using filterless UVA/UVC technology to prevent viral spread in a localized volume. For example, the low-form table-top air sanitizer can be used where people are indoors, in close proximity, and not wearing masks. Also described herein is a method of using a low-form table-top air sanitizer to purify air in an indoor, close proximity environment where wearing a mask is impractical.

### CROSS-REFERENCE TO RELATED APPLICATIONS

This patent application claims priority to and filing benefit of U.S. Provisional Patent Application No. 63/081,499 filed Sep. 22, 2020, entitled, “AIR-EXCHANGING SANITATION APPARATUS AND METHOD,” and naming Peter R. Nuytkens, Mark H. Nuytkens, Amy R. Nuytkens, and Margaret H. Nuytkens as inventors, the disclosure of which is incorporated herein by reference in its entirety.

### TECHNICAL FIELD

Illustrative embodiments of the invention generally relate to air sanitization, and, more particularly, various embodiments of the invention relate to removing airborne contaminants using a reduced form factor.

### BACKGROUND

International economic health and personal quality of life have been drastically compromised due to social (physical) distancing requirements to help stop the spread of viruses and other pathogens to keep people safe. There is a need for personal space sanitization of aerosolized contaminants.

### SUMMARY

In accordance with one embodiment of the invention, described herein is an air treatment system, comprising: a housing forming an inlet for receiving exterior air and an outlet for expelling treated air; a channel between the inlet and the outlet; and an air sanitizer within the channel and configured to apply UVA and UVC light to air traversing between the inlet and the outlet. In certain embodiments, the channel forms a tortuous path between the inlet and the outlet (e.g., a helical path, a spiral path, or a serpentine path). In certain embodiments, the air treatment system comprises an air moving device configured to draw air into the inlet and expel treated air through the outlet. In some embodiments, the housing and the air moving device are configured to produce an air vortex exterior to the housing. In some cases, the channel is positioned radially about the air moving device. According to certain embodiments described herein, the air sanitizer is configured to kill 99 percent (%) to 99.9% of pathogens in exterior air entering through the inlet. As described herein, the air sanitizer comprises an array of light emitting diodes. In some cases, the system produces an output noise of from 1 to 30 decibels (dB). In some embodiments, the air treatment system described herein includes logic configured to produce an output flow of treated air at a rate of between 3 and 7 liters per second.

Also described herein is a method of treating air, comprising: receiving exterior air into a housing comprising an air sanitizer positioned within a tortuous channel; flowing the received exterior air through the tortuous channel and exposing the received exterior air to UVA and UVC light to provide treated air; and expelling the treated air to a surrounding environment. In some embodiments, flowing the received exterior air through the tortuous channel comprises flowing the received exterior air through a tortuous channel having a length of from 75 centimeters (cm) to 1.5 meters (m). In some embodiments, flowing the received exterior air through the tortuous channel comprises flowing the received exterior air through the tortuous channel at a rate of from at least 3 liters per second (L/s) up to 7 L/s (e.g., at a rate of at least 5 L/s). In some cases, exposing the received exterior air to UVA and UVC lights comprises destroying a virus. Destroying a virus comprises generating pyrimidine dimers and oxidized bases by exposing the virus to UVA light and cleaving a DNA strand by exposing the virus to UVC light. As described herein, exposing the virus to UVA and UVC light does not produce ozone (O₃). In some embodiments, the method described herein comprises exposing 5 liters (L) of received exterior air to at least 1 milliJoule (mJ) per square centimeter (cm²) of UVA and UVC light for from 0.25 seconds (s) to 2 s. Additionally, the method described herein comprises outputting less than 30 dB of noise.

Also described herein is an air treatment system, comprising: a housing comprising an inlet and an outlet connected by a 360° mirrored tortuous channel, wherein the housing comprises a volume of up to 1500 cubic centimeters (cm³); an air movement system configured to draw exterior air into the inlet, urge the air through the 360° mirrored tortuous channel, and expel the air though the outlet at a rate of at least 3 L/s; an air treatment system configured to expose the air in the 360° mirrored tortuous channel to at least 1 mJ/cm² of UVA and UVC light; a rechargeable power supply configured to provide at least 5 hours (h) of continuous service; and a noise output of less than 30 dB. In some embodiments, the housing is configured to expel air in a toroidal vortex, wherein the toroidal vortex provides a barrier preventing exhaled breath flow from at least a first subject positioned adjacent to the air treatment system to at least a second subject positioned adjacent to the air treatment system.

Covered embodiments of the invention are defined by the claims, not this summary. This summary is a high-level overview of various embodiments of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification, any or all drawings, and each claim.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIG. 1 schematically shows a 3D view of an assembly of an air treatment system in accordance with illustrative embodiments of the invention.

FIG. 2 schematically shows a cross-sectional, exploded front half wire frame view of the personal air cleaner of FIG. 1.

FIG. 3 schematically shows a cross-sectional, exploded transparent wire frame view of the personal air cleaner of FIG. 1.

FIG. 4 schematically shows a cross-sectional, assembled transparent wire frame view of the personal air cleaner of FIG. 1.

FIG. 5 schematically shows a cross-sectional, assembled and rendered 3D view of the personal air cleaner of FIG. 1.

FIG. 6 shows a system diagram of the air treatment system of FIG. 1.

FIG. 7 schematically illustrates a sanitizing channel within the air treatment system of FIG. 1.

FIG. 8 is a graph showing efficacy of the air treatment system of FIG. 1.

FIG. 9 is a graph showing efficacy of the air treatment system of FIG. 1.

### DETAILED DESCRIPTION

According to the Centers for Disease Control (CDC) and the National Foundation for Infectious Diseases (NFID), SARS-CoV-2, the virus that causes COVID-19, is spread mainly through respiratory droplets transmitted from person to person by breathing, talking, and coughing within about 6 feet of each other. The droplets vary in size, and as they are exhaled and move away from their source they are suspended in the air for seconds to minutes. The droplets diffuse into the larger volume of air they encounter. Recent studies have indicated that COVID-19 may be spread by people who are not showing symptoms, which is why face masks are recommended for all people during the COVID-19 pandemic.

Based on the above viral transmission information from the CDC, it logically follows that preventing viral spread in scenarios where people are indoors, in close proximity, and not wearing masks requires a drastically different approach. A low-form, table-top air sanitizer can significantly reduce the chances of viral transmission. As such, the low-form table-top air sanitizer described herein, by continuously generating a dominant sterile air current, can interrupt local air exchanges between persons sitting at a table, and can reduce and dilute any viral load in the local air volume. Application of such a device can enable social, lifestyle, and business activities to resume with increased safety, e.g., eating in restaurants where masks are removed. The use case can be extended to include engaging in face-to-face business transactions across a desk such as in a bank, office, or educational setting, as well as all manner of small-group round-table meetings, and traveling on airplanes, trains, carpools, and/or taxis. The salient design features of an optimum table-top air sanitizer are that it must be effective, safe, simple, portable, quiet, elegant, and tested. The following sections will expand upon each of these features in more detail.

As stated above, the air sanitizer includes UVA and UVC light sources, particularly, high-intensity UVA and UVC multi-emitter sources (e.g., light emitting diode (LED) strips). The air sanitizer can also include LEDs emitting light in a plurality of UVA and UVC wavelengths (e.g., wavelengths ranging from 190 nm to 400 nm). Optionally, the air sanitizer can include a mirrored 360° tortuous channel (i.e., a multi-angle exposure channel) configured to channel internal reflection to ensure viral exposure to UVA and/or UVC light.

It is well known in the art that ultraviolet (UV) light induces damage to the genomes of bacteria, protozoa, and viruses. UV light can break molecular bonds and form photodimeric lesions in nucleic acids (e.g., DNA and RNA). These lesions prevent both transcription and replication and ultimately lead to inactivation of the microorganisms. As incorporated herein, UV light technology is effective, safe, convenient, and inexpensive. Particularly, the UV light described herein provides a filterless air treatment system. Further, the UV light described herein is emitted at wavelengths that fail to produce O₃.

Not to be bound by theory, nucleic acid lesions prevent genetic replication. Pyrimidine dimer (PD) is a well known DNA lesion affecting a single DNA strand. It is an intrastrand cross-link, in which two adjacent pyrimidines are connected by a cyclobutane ring. PDs are induced in DNA by UV light. The short-wavelength UV light (190-280 nm, UVC) yields mostly PDs, while mid-wavelength UV light (280-320 nm, UVB) and especially long-wavelength UV (320-400 nm, UVA) yield a mixture of PDs and oxidized bases. UVA can penetrate farther into organic material than UVC due to its longer wavelength. Exposure to UVA light allows any genetic material in the virus and/or pathogen to be exposed to the UVA and UVC light emitted from the air sanitizer. Thus, multi-wavelength UV light sources comprising broad band UVC and UVA are more efficient and require less power for effective sterilization by disrupting viral and/or pathogenic replication.

Accordingly, damage to viruses and/or pathogens caused by UV light occurs when the UV light is emitted at wavelengths absorbed by DNA and RNA, e.g., in the UV germicidal irradiation region between 200 nm and 300 nm. Inactivating virus-containing aerosols by UV germicidal irradiation requires significantly less UV dosage than for other human contaminants, e.g., bacteria or mold spores. The very simple structure of a virus limits the absorption and shielding of UV energy such that the photonic energy is not scattered and directly transfers energy to the genetic material causing lesion. Coronavirus SARS-CoV-2 has a simple ssRNA structure and when in aerosolized form and subjected to line-of-sight photonic bombardment, it requires less than 1 mJ/cm² to obtain at least 99% virus inactivation. In some cases, viruses embedded on a surface may require 50 mJ/cm² to achieve similar levels of inactivation.

The ability of UVC and UVA light irradiation to inactivate viruses depends on a line-of-sight pathway from source to target. Viruses and/or pathogens on a surface can be embedded in the surface and blocked by surface topography necessitating an order of magnitude higher dosage when compared to aerosolized contagions. Aerosolized particles have no surface absorption and scattering barriers making aerosolized particles vulnerable from an all-angle attack. As described herein, an optimum exposure channel includes multiple emitter sources with a wide-angle emission arranged in spatial arrays directed into a reflecting enclosed channel (e.g., a 360° mirrored tortuous channel). In such a channel, photons bounce multiple times providing multiple paths and passes that provide multiple interactions with virus and/or pathogen targets. These combined features form an irradiation channel requiring less UV germicidal irradiation dosage, thereby increasing sterilization efficacy.

Particularly, UVC light having a wavelength in the range of 100 nm to 270 nm is optimum for killing viruses, bacteria, and mold spores. These wavelengths coincide with the peak absorption bandwidths of chemical bonds in the genetic material (e.g., DNA and RNA) of viruses, bacteria, and mold spores. UVC exposure causes DNA either to change shape or be physically damaged (e.g., cleaved) rendering it unable to replicate in a continuous manner. In certain cases, the UVC optical energy density required to split DNA of viruses on a surface ranges from 10 mJ/cm² to 100 mJ/cm² (e.g., from 15 mJ/cm² to 95 mJ/cm², from 17 mJ/cm² to 97 mJ/cm², from 20 mJ/cm² to 90 mJ/cm², from 11 mJ/cm² to 100 mJ/cm², from 10 mJ/cm² to 99 mJ/cm², or from 11 mJ/cm² to 99 mJ/cm²). Aerosolized viruses and/or pathogens require a substantially smaller dosage since there is no surface field or topography inhibiting exposure.

Additional to destroying viral and pathogenic species in the air drawn into the air treatment system, the system can create a barrier that restricts breath transmission from at least a first human subject and at least a second human subject by providing a toroid vortex above the air treatment system that interrupts air flow between at least the first human subject and at least the second human subject. For example, the air treatment system can provide localized protection in a proximal setting encompassing about 1 cubic meter (m³) of space. Within the 1 m³, the air treatment system described herein can treat the 1 m³ of air in about 5 seconds. Finally, the air treatment system described herein can create a continuous local toroidal vortex barrier between human subjects positioned about the air treatment system. For example, the air treatment system described herein can provide a protective sanitized bubble for the human subjects positioned about the air treatment system. As such, a sterile air supply fails to fully protect human subjects in close proximity against airborne viruses and/or pathogens. Where a sterile air supply provides an initially sterile environment, human subjects instantly desterilize the air by exhaling. After exhaling, airflow trajectories within the proximal environment affect portability of airborne viruses and/or pathogens. Localized air currents caused by subjects and/or objects in the environment (e.g., exhaled breath, HVAC supply, moving bodies, heat sources, cold sinks, fans, and the like) contribute to particle interactions of potentially contaminated aerosols present in the air. Currents generated locally by respiratory activity are dominant compared to global air turnover from HVAC units operating by diffusion. Turning over the volume of air in the entire room is less effective and far less efficient then sterilizing the local air continuously where people spend the majority of their time breathing in close proximity. Providing a source of sterile air among multiple people sitting at a table creates a locally reduced viral concentration zone and reduces particle interactions from one person to another.

In illustrative embodiments, the air treatment system effectively sanitizes air with a smaller form factor. For example, the air treatment system described herein is a table-top device having dimensions that provide a displacement volume of no more than 1500 cm³. The air treatment system described herein provides an air treatment pathway that is a tortuous path guiding air through sanitizing regions (e.g., UVA and UVC light exposure) via a spiral shaped sanitizing region.

In certain embodiments, air drawn into the inlet (e.g., received exterior air) traverses the tortuous channel at a rate of from at least 3 liters per second (L/s) up to 7 L/s (e.g., from 3.1 L/s to 7 L/s, from 3 L/s to 6.9 L/s, from 3.1 L/s to 6.9 L/s, from 3.5 L/s to 6.5 L/s, from 4 L/s to 6 L/s, from 4.5 L/s to 5.5 L/s, or about 5 L/s). For example, the received exterior air can traverse the tortuous channel at a rate of 3.1 L/s, 3.2 L/s, 3.3 L/s, 3.4 L/s, 3.5 L/s, 3.6 L/s, 3.7 L/s, 3.8 L/s, 3.9 L/s, 4.0 L/s, 4.1 L/s, 4.2 L/s, 4.3 L/s, 4.4 L/s, 4.5 L/s, 4.6 L/s, 4.7 L/s, 4.8 L/s, 4.9 L/s, 5.0 L/s, 5.1 L/s, 5.2 L/s, 5.3 L/s, 5.4 L/s, 5.5 L/s, 5.6 L/s, 5.7 L/s, 5.8 L/s, 5.9 L/s, 6.0 L/s, 6.1 L/s, 6.2 L/s, 6.3 L/s, 6.4 L/s, 6.5 L/s, 6.6 L/s, 6.7 L/s, 6.8 L/s, 6.9 L/s, or 7.0 L/s.

Analysis of an exemplary personal space air sanitizer in an enclosed space (e.g., on a restaurant table) is as follows: The average adult inhales and exhales air at a rate of about 7 L/min (0.25 cubic feet per minute (CFM)). For example, close proximity air use for a typical one square meter area dining table of 4 adults can provide human air exchange at a rate of about 28 L/min (1 CFM). Effective air sanitizing can be performed by cleaning the air at a rate of 2 to 10 times the human air exchange rate (e.g., 56 to 280 L/min). Cleaning the air at a rate greater than the human air exchange rate reduces the virus and/or pathogen concentration of aerosolized contaminants in the air, and may reduce the concentration of larger contaminants as well. Additionally, this rate can mitigate the rate of diffusion of exhaled aerosols, which continuously expand into the surrounding volume of air.

In illustrative embodiments, a sanitizing pathway is designed using a UVC 254 nm LED strip including 60 diodes per meter spaced 1.6 cm from center to center of each LED. Each LED emits an optical power of 240 milliwatts (mW) into a side-, top-, and bottom-reflecting spiral channel 4 cm wide by 1 cm high by 100 cm (1 m) long. This produces a 1 m long air-sanitizing region or channel (e.g., a 1 m tortuous path) having a volume of at least 400 cm³.

In some cases, an air flow rate of about 28 L/min (1 CFM)=472 cm³/s (0.472 L/s) produces an inlet to outlet channel transit time of about 1 s at 144 mJ/cm² of UVC exposure to continuously clean the volume of air traversing the channel. Exposure dosage (mJ/cm²)=UV Irradiance (mW/cm²)×Time (s). This 1 s transit time of sterilization exposure is in excess of the FDA and CDC recommended 10 mJ/cm² to 100 mJ/cm² surface dose for virus, bacteria, and mold spore sanitation, and is greater than 10 times the required free air volume sterilization dose. It should be noted that these specific parameters and numbers are illustrative of a specific example, and those skilled in the art can apply these examples to various embodiments.

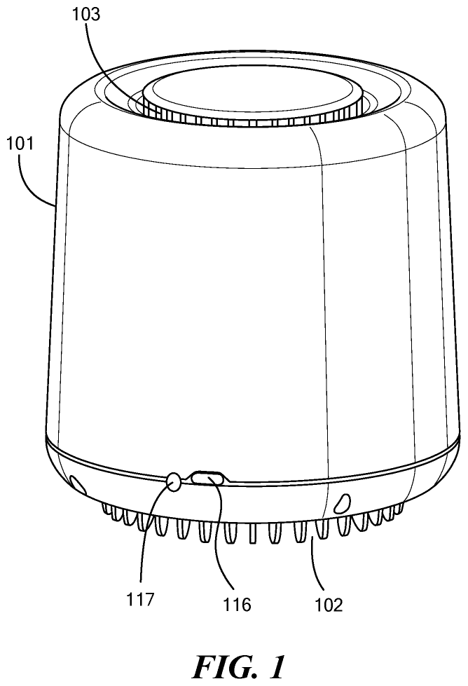

Turning now to the drawings, FIGS. 1-5 schematically show an air treatment system configured in accordance with illustrative embodiments. The sanitizer of FIGS. 1-5 is configured as an assembly including a housing containing and forming a plurality of components. Among other things, those components include a base inlet duct 102 for receiving inlet air, a forced air optical sanitizing flow channel 104 for sanitizing air received through the inlet duct 102, and an outlet duct 103 for discharging cleaned/sanitized air.

In certain embodiments, air is drawn into the housing through fluted radial symmetric openings of the inlet duct 102, and into and through a flow channel 104 that includes a plurality of sanitizing UVA and UVC light emitters 109, although other embodiments may use other cleaning modalities. The air flow is preferably drawn into the device at a constant air flow rate, creating a constant optical radiation channel (e.g., tortuous path) transit exposure time, and then exits out of the housing fluted radial symmetric openings of the outlet duct 103. In some examples, the air treatment system receives power through a USB C charge port 116 or from an internal rechargeable battery 106 with operation on/off control through a power button 117. Optionally, the device may receive power from an AC outlet.

As described herein, the forced air optical sanitizing device flow channel 104 is optimized and configured to meet or exceed certain air cleaning requirements. Preferably, its length, cross sectional area, flow rate, UVA and UVC power density (mW/cm³) are optimized to exceed FDA in-air dose sterilization requirements in terms of energy required for space and radiant fluence (J/m²) across a cross-section of a UVC beam. Moreover, the flow channel 104 is configured in a tortuous path, such as a spiral, serpentine, helix, or other shape to enable sufficient time for air to be exposed to the sanitization/cleaning LEDs.

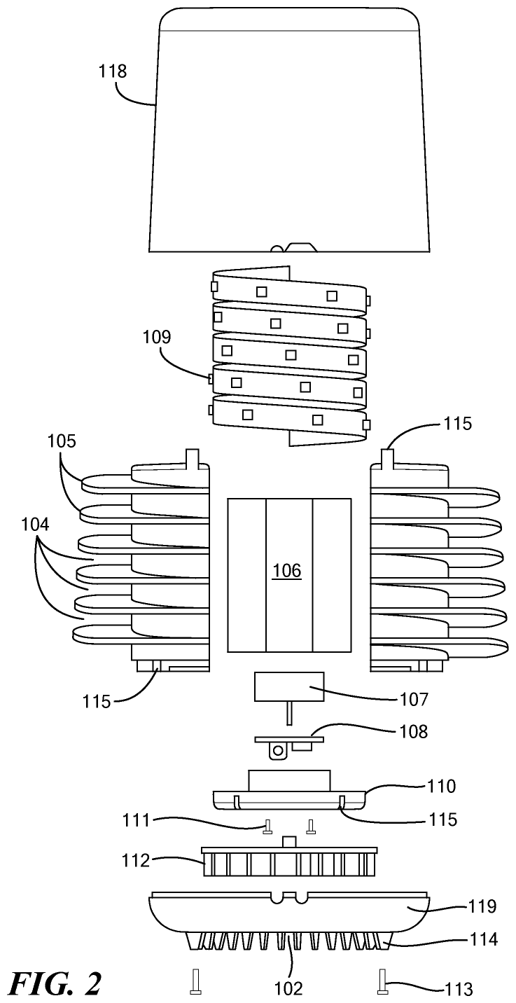

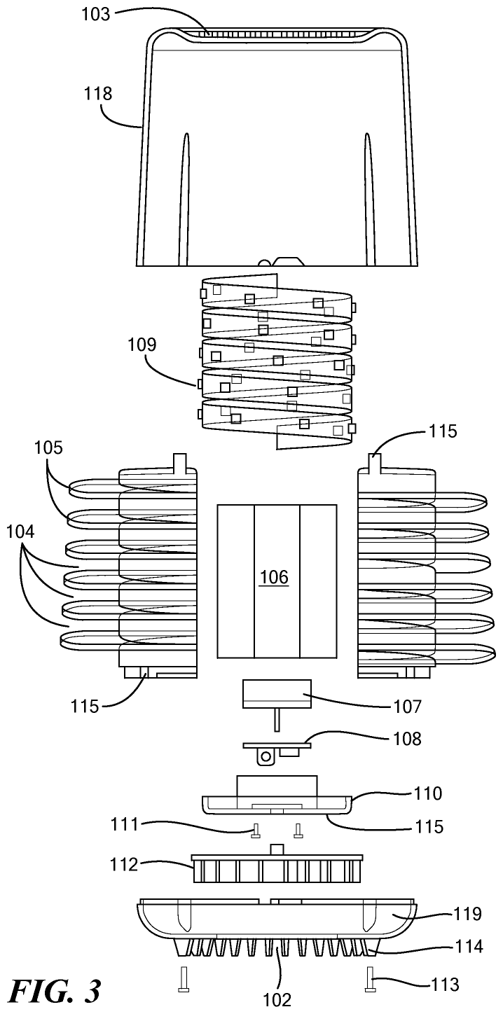

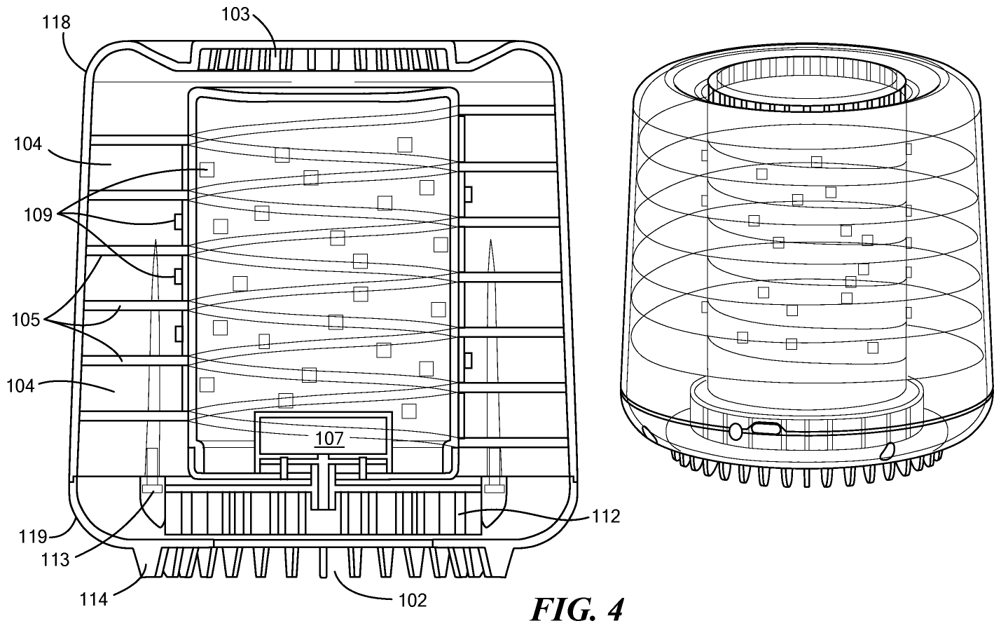

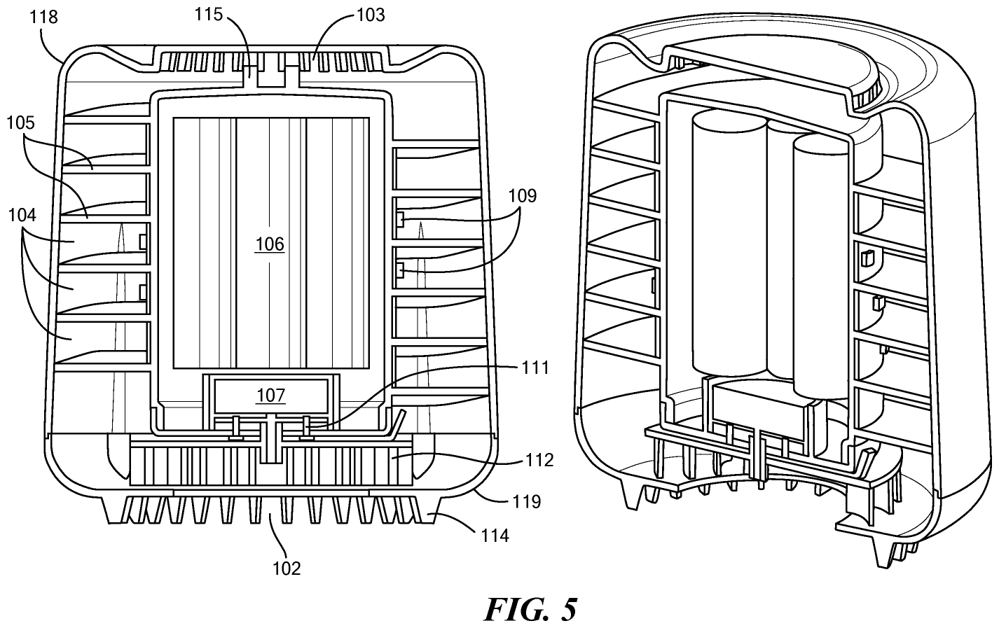

FIG. 2 schematically shows a cross-sectional exploded front half wire frame and view of the air treatment system. This view expressly shows the flow channel 104, a spiral flange 105, a rechargeable battery 106, a motor 107 (e.g., a brushless DC motor) to urge air flow, a controller printed circuit board 108, one or more UVA and/or UVC LEDs 109, a motor bracket 110 and motor mounting screws 111, an impeller 112 coupled with the stator of the motor to produce air flow, and housing screws 113, support feet 114, barbed snap locking features 115, a top shell 118, and a base shell 119 forming the housing. FIG. 3 schematically shows a cross-section exploded transparent wire frame view of the device, detailing the inlet duct 102, the outlet duct 103, the flow channel 104, the spiral flange 105, the rechargeable battery 106, the motor 107, the controller printed circuit board 108, the UVA and/or UVC LEDs 109, the impeller 112, and the motor bracket 110, motor mounting screws 111, housing screws 113, support feet 114, barbed snap locking features 115, top shell 118, and base shell 119. FIG. 4 schematically shows the assembled transparent wire frame view of the device including the inlet duct 102, the outlet duct 103, the flow channel 104, the spiral flange 105, the UVA and/or UVC LEDs 109, and the housing screws 113, support feet 114, top shell 118, and base shell 119. FIG. 5 schematically shows a cross-sectional, assembled view of the device including the inlet duct 102, the outlet duct 103, the flow channel 104, the spiral flange 105, the rechargeable battery 106, the motor 107, the UVA and/or UVC LEDs 109, the impeller 112, and the motor mounting screws 111, support feet 114 barbed snap locking features 115, top shell 118, and base shell 119.

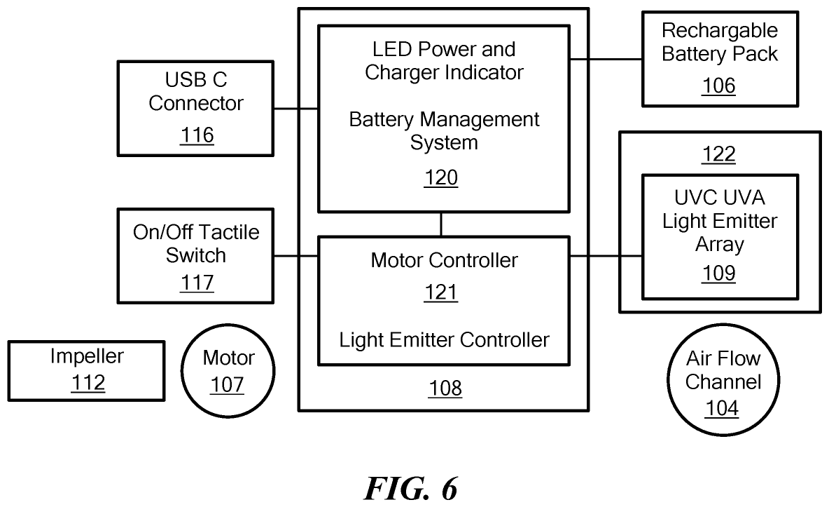

FIG. 6 schematically shows a system diagram of the device including the USB C charge port 116 power and control connector, a tactile on/off power button 117, a battery management system 120, the rechargeable battery 106, the controller printed circuit board 108, a motor controller 121 to control operation of the motor 107, a LED controller 122 to control UVA and/or UVC LEDs 109, the UVA and/or UVC LEDs 109, the flow channel 104, the motor 107, and the impeller 112.

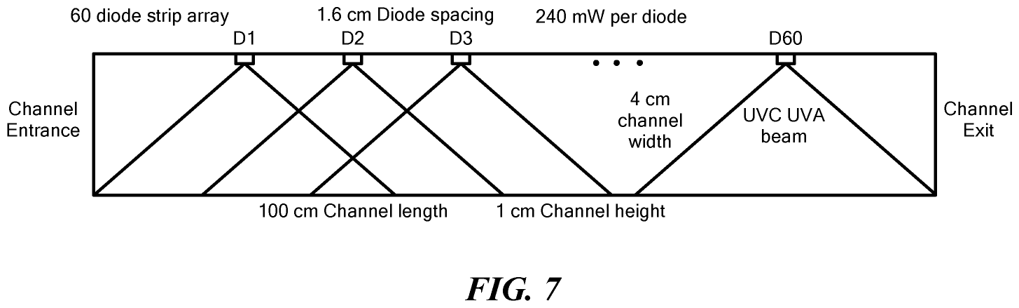

FIG. 7 schematically shows an exemplary sanitizing channel design using a UVC 254 nm LED strip having 60 diodes per meter emitting UVC at 240 mW spaced 1.6 cm center to center. The LEDs emit the UVC into the side-, top- and bottom-reflecting channel (e.g., 360° mirrored channel) that is 4 cm wide by 1 cm high by 100 cm (1 m) long providing a sanitization volume of 400 cm³.

In some cases, the air treatment system 101 contains an internal flow channel 104 having a plurality of UVA and/or UVC LEDs 109 continuously connected from the inlet duct 102 to the outlet duct 103, through which air is exposed for a sufficient duration to sterilize viruses, bacteria and mold spores that may be present in vaporous or particulate aerosols.

The flow of air into and out of the device is achieved using either the impeller 112 or a blower. Preferably, the inlet and outlet are ducted or open on opposite extremities of the device to minimize mixing of inlet and outlet air flows. Moreover, the outlet flow preferably is directed in a uniformly radial direction away from the device's central vertical axis and slightly upward in a compact optimum inter channel mixing optical exposure.

The device also has a brushless DC three-phase motor that runs at a high torque and at a low radial speed, enabling the radial impeller blades generate air flow while turning at low radial speed. Accordingly, the dominant acoustic noise spectrum shifts to a lower frequency range resulting in less audible noise. For example, the air treatment system produces an acoustic power below 40 dBA while still generating a 28 to 280 L/min flow rate. The motor controller may add dither noise and adjust the motor radial speed to break resonance with the natural frequencies of the channel and device components.

For cleaning, the air treatment system may have a UVC optical spectrometer assessing the load of DNA and/or RNA in the air entering the sterilization channel. Among other things, the concentration measurement output may be displayed via a pulse width modulated light indicator or digital display indicator, measuring the fidelity of the air flow sterilization. In certain cases, the air treatment system includes a visible LED lighted feature that provides ambient lighting to the surrounding environment. A wireless connectivity modality, such as a blue-tooth controlled speaker and microphone, also may be included. Moreover, the noted USB C connector can charge external devices, such as a mobile telephone or computer device.

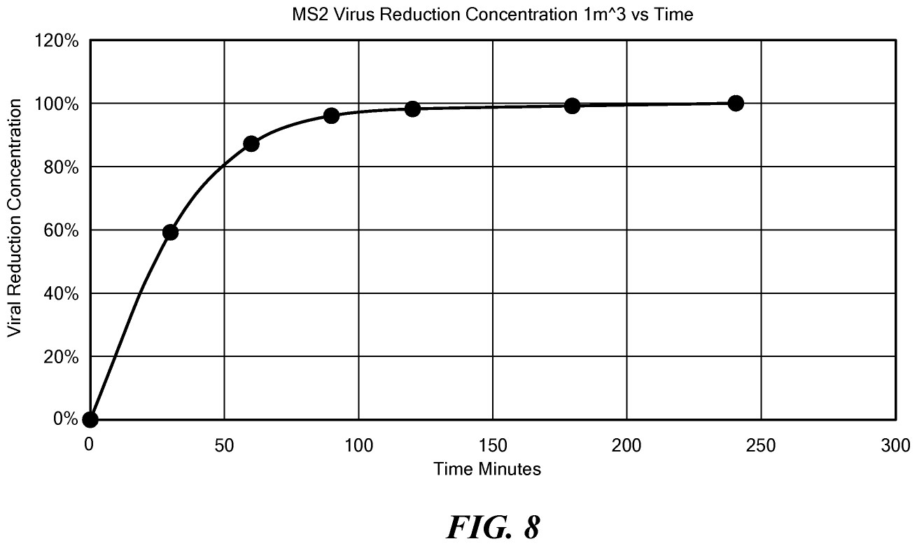

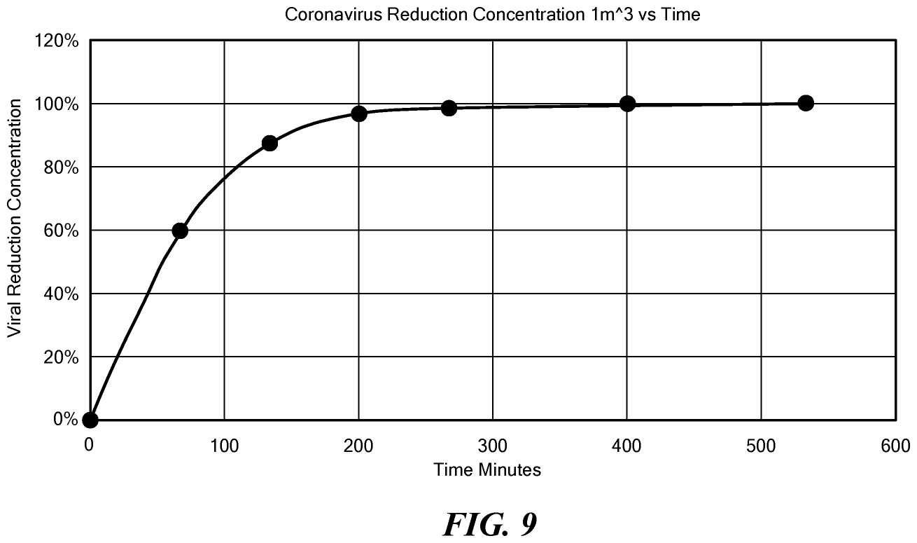

FIGS. 8-9 show the efficacy of the air treatment system in reducing viral concentration of a 1 m³ (1000 L) localized volume. FIG. 8 shows a reduction of the MS2 virus over a time of up to 250 s. As shown in FIG. 8, the concentration of the MS2 virus occupying a 1 m³ volume was reduced 99.8% after a UVA and UVC LED exposure dose of 54 mJ/cm² in 240 minutes. FIG. 9 shows a reduction of the SARS-CoV-2 virus over a time of up to 525 s. As shown in FIG. 9, the concentration of the SARS-CoV-2 virus occupying a 1 m³ volume was reduced 99.9% after a UVA and UVC LED exposure dose of 2 mJ/cm² in 400 seconds. The air treatment system described herein kills the coronavirus in a single pass at a flow rate of 5 L/sec.

Accordingly, the air treatment system preferably is portable and has a small form factor, while providing significant air cleaning capabilities. In certain embodiments, the air treatment system has a volume displacement of up to 1500 cm³.

### Definitions and Descriptions

As used herein, the terms “invention,” “the invention,” “this invention,” and “the present invention” are intended to refer broadly to all of the subject matter of this patent application and the claims below. Statements containing these terms should be understood not to limit the subject matter described herein or to limit the meaning or scope of the patent claims below.

As used herein, the meaning of “a,” “an,” and “the” includes singular and plural references unless the context clearly dictates otherwise.

As used herein, the meaning of “room temperature” can include a temperature of from about 15° C. to about 30° C., for example about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., or about 30° C.

All ranges disclosed herein are to be understood to encompass any and all endpoints as well as any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g. 1 to 6.1, and ending with a maximum value of 10 or less, e.g., 5.5 to 10.

The term “and/or” when used in a list of two or more items, means that any one of the listed items can be employed by itself or in combination with any one or more of the listed items. For example, the expression “A and/or B” is intended to mean either or both of A and B, i.e., A alone, B alone, or A and B in combination. The expression “A, B and/or C” is intended to mean A alone, B alone, C alone, A and B in combination, A and C in combination, B and C in combination or A, B, and C in combination.

The embodiments of the invention described above are intended to be merely exemplary; numerous variations and modifications will be apparent to those skilled in the art. Such variations and modifications are intended to be within the scope of the present invention as defined by any of the appended claims.

### Iterations of Select Embodiments

Iteration 1 is an air treatment system, comprising: a housing forming an inlet for receiving exterior air and an outlet for expelling treated air; a channel between the inlet and the outlet; and an air sanitizer within the channel and configured to apply UVA and UVC light to air traversing between the inlet and the outlet.

Iteration 2 is the air treatment system of any preceding or subsequent iteration, wherein the channel forms a tortuous path between the inlet and the outlet.

Iteration 3 is the air treatment system of any preceding or subsequent iteration, wherein the tortuous path comprises a helical path, a spiral path, or a serpentine path.

Iteration 4 is the air treatment system of any preceding or subsequent iteration, comprising an air moving device configured to draw air into the inlet and expel treated air through the outlet.

Iteration 5 is the air treatment system of any preceding or subsequent iteration, wherein the housing and the air moving device are configured to produce an air vortex exterior to the housing.

Iteration 6 is the air treatment system of any preceding or subsequent iteration, wherein the channel is positioned radially about the air moving device.

Iteration 7 is the air treatment system of any preceding or subsequent iteration, wherein the air sanitizer is configured to kill 99 percent to 99.9 percent of pathogens in exterior air entering through the inlet.

Iteration 8 is the air treatment system of any preceding or subsequent iteration, wherein the air sanitizer comprises an array of light emitting diodes.

Iteration 9 is the air treatment system of any preceding or subsequent iteration, wherein the system produces an output noise of from 1 to 30 dB.

Iteration 10 is the air treatment system of any preceding or subsequent iteration, comprising logic configured to produce an output flow of treated air at a rate of between 3 and 7 liters per second.

Iteration 11 is a method of treating air according to the air treatment system of any preceding or subsequent iteration, comprising: receiving exterior air into a housing comprising an air sanitizer positioned within a tortuous channel; flowing the received exterior air through the tortuous channel and exposing the received exterior air to UVA and UVC light to provide treated air; and expelling the treated air to a surrounding environment.

Iteration 12 is the air treatment system of any preceding or subsequent iteration, wherein flowing the received exterior air through the tortuous channel comprises flowing the received exterior air through a tortuous channel having a length of from 75 centimeters to 1.5 meters.

Iteration 13 is the air treatment system of any preceding or subsequent iteration, wherein flowing the received exterior air through the tortuous channel comprises flowing the received exterior air through the tortuous channel at a rate of from at least 3 liters per second up to 7 liters per second.

Iteration 14 is the air treatment system of any preceding or subsequent iteration, wherein flowing the received exterior air through the tortuous channel comprises flowing the received exterior air through the tortuous channel at a rate of at least 5 liters per second.

Iteration 15 is the air treatment system of any preceding or subsequent iteration, wherein exposing the received exterior air to UVA and UVC lights comprises destroying a virus.

Iteration 16 is the air treatment system of any preceding or subsequent iteration, wherein destroying a virus comprises generating pyrimidine dimers and oxidized bases by exposing the virus to UVA light and cleaving a DNA strand by exposing the virus to UVC light.

Iteration 17 is the air treatment system of any preceding or subsequent iteration, wherein exposing the virus to UVA and UVC light does not produce ozone.

Iteration 18 is the air treatment system of any preceding or subsequent iteration, comprising exposing 5 liters of received exterior air to at least 1 milliJoule per square centimeter of UVA and UVC light for from 0.25 seconds to 2 seconds.

Iteration 19 is the air treatment system of any preceding or subsequent iteration, comprising outputting less than 30 dB of noise.

Iteration 20 is the air treatment system of any preceding or subsequent iteration, comprising: a housing comprising an inlet and an outlet connected by a 360° mirrored tortuous channel, wherein the housing comprises a volume of up to 1500 cubic centimeters; an air movement system configured to draw exterior air into the inlet, urge the air through the 360° mirrored tortuous channel, and expel the air though the outlet at a rate of at least 3 liters per second; an air treatment system configured to expose the air in the 360° mirrored tortuous channel to at least 1 milliJoule per square centimeter of UVA and UVC light; a rechargeable power supply configured to provide at least 5 hours of continuous service; and a noise output of less than 30 dB.

Iteration 21 is the air treatment system of any preceding or subsequent iteration, wherein the housing is configured to expel air in a toroidal vortex.

Iteration 22 is the air treatment system of any preceding iteration, wherein the toroidal vortex provides a barrier preventing exhaled breath flow from at least a first subject positioned adjacent to the air treatment system to at least a second subject positioned adjacent to the air treatment system.

All patents, publications, and abstracts cited above are incorporated herein by reference in their entireties. Various embodiments of the invention have been described in fulfillment of the various objectives of the invention. It should be recognized that these embodiments are merely illustrative of the principles of the present invention. Numerous modifications and adaptions thereof will be readily apparent to those skilled in the art without departing from the spirit and scope of the present invention as defined in the following claims.

What is claimed is:

1. An air treatment system, comprising:
a housing forming an inlet for receiving exterior air and an outlet for expelling treated air;
a channel between the inlet and the outlet; and
an air sanitizer within the channel and configured to apply UVA and UVC light to air traversing between the inlet and the outlet.

2. The air treatment system of claim 1, wherein the channel forms a tortuous path between the inlet and the outlet.

3. The air treatment system of claim 2, wherein the tortuous path comprises a helical path, a spiral path, or a serpentine path.

4. The air treatment system of claim 1, comprising an air moving device configured to draw air into the inlet and expel treated air through the outlet.

5. The air treatment system of claim 4, wherein the housing and the air moving device are configured to produce an air vortex exterior to the housing.

6. The air treatment system of claim 4 wherein the channel is positioned radially about the air moving device.

7. The air treatment system of claim 1, wherein the air sanitizer is configured to kill 99 percent to 99.9 percent of pathogens in exterior air entering through the inlet.

8. The air treatment system of claim 1, wherein the air sanitizer comprises an array of light emitting diodes.

9. The air treatment system of claim 1, wherein the system produces an output noise of from 1 to 30 dB.

10. The air treatment system of claim 1, comprising logic configured to produce an output flow of treated air at a rate of between 3 and 7 liters per second.

11. A method of treating air, comprising:
receiving exterior air into a housing comprising an air sanitizer positioned within a tortuous channel;
flowing the received exterior air through the tortuous channel and exposing the received exterior air to UVA and UVC light to provide treated air; and
expelling the treated air to a surrounding environment.

12. The method of claim 11, wherein flowing the received exterior air through the tortuous channel comprises flowing the received exterior air through a tortuous channel having a length of from 75 centimeters to 1.5 meters.

13. The method of claim 11, wherein flowing the received exterior air through the tortuous channel comprises flowing the received exterior air through the tortuous channel at a rate of from at least 3 liters per second up to 7 liters per second.

14. The method of claim 13, wherein flowing the received exterior air through the tortuous channel comprises flowing the received exterior air through the tortuous channel at a rate of at least 5 liters per second.

15. The method of claim 11, wherein exposing the received exterior air to UVA and UVC lights comprises destroying a virus.

16. The method of claim 15, wherein destroying a virus comprises generating pyrimidine dimers and oxidized bases by exposing the virus to UVA light and cleaving a DNA strand by exposing the virus to UVC light.

17. The method of claim 15, wherein exposing the virus to UVA and UVC light does not produce ozone.

18. The method of claim 11, comprising exposing 5 liters of received exterior air to at least 1 milliJoule per square centimeter of UVA and UVC light for from 0.25 seconds to 2 seconds.

19. The method of claim 11, comprising outputting less than 30 dB of noise.

20. An air treatment system, comprising:
a housing comprising an inlet and an outlet connected by a 360° mirrored tortuous channel, wherein the housing comprises a volume of up to 1500 cubic centimeters;
an air movement system configured to draw exterior air into the inlet, urge the air through the 360° mirrored tortuous channel, and expel the air though the outlet at a rate of at least 3 liters per second;
an air treatment system configured to expose the air in the 360° mirrored tortuous channel to at least 1 milliJoule per square centimeter of UVA and UVC light;
a rechargeable power supply configured to provide at least 5 hours of continuous service; and
a noise output of less than 30 dB.

21. The air treatment system of claim 20, wherein the housing is configured to expel air in a toroidal vortex.

22. The air treatment system of claim 21, wherein the toroidal vortex provides a barrier preventing exhaled breath flow from at least a first subject positioned adjacent to the air treatment system to at least a second subject positioned adjacent to the air treatment system.
